(19) United States
(12) Patent Application Publication
(10) Pub. No.: US 2009/0131973 A1
(43) Pub. Date: May 21, 2009

(54) Device for relieving pain or tension

(76) Inventor: Melody Selene Zacharias, Kelowna (CA)

Correspondence Address: Bull Housser & Tupper, 3000 Royal Centre, 1055 West Georgia Street, Vancouver, BC, V6E 3R3, CA

(21) Appl. No.: 12/289,821

(22) Filed: Nov. 5, 2008

Related U.S. Application Data
(60) Provisional application No. 60/985,569, filed on Nov. 5, 2007.

(51) Int. Cl. A61B 17/00; A61G 15/00; A61F 5/00

(52) U.S. Cl. 606/204; 128/845; 606/240

(57) ABSTRACT

A device for relieving pain and tension is provided. The device includes a base having at least one pressure member support on a first side of the base and at least one pressure member extending upwardly and rearwardly relative to the pressure member support wherein the pressure member is configured to engage a user. For use in association with the neck a and head region, preferably, the device includes two pressure members positioned on the pressure member support such that the pressure members may support the head of user while engaging the occipital region of the user's head.

### PRIORITY CLAIM

The current application is a formal application of U.S. Patent Application No. 60/985,569 entitled A DEVICE FOR RELIEVING THE PAIN ASSOCIATED WITH THE CONDITION OR CONDITIONS COMMONLY REFERRED TO AS A HEADACHE filed on the 5 Nov. 2007.

### FIELD OF INVENTION

The present invention relates generally to a support device, and more particularly, it relates to a support device configured to receive the head of a user for therapeutic and tension and pain relief purposes.

### BACKGROUND OF THE INVENTION

Virtually everyone has experienced a headache before, regardless of age or gender. Of all those headache sufferers, there are many who regularly live with pain or discomfort in the head, scalp, or neck region. Although there are many medical explanations for such pain or discomfort, headaches are typically associated with muscle tightness and/or poor circulation. Headaches occur when neck and head muscles become tense or contract, for example, in response to stress, depression, head injury, or anxiety. Muscle tightness and contraction may cause constriction of blood flow which usually results in the onset of a headache. Furthermore, everyday activities that require the head to be held in one position for a long time without moving, for example, when typing or working on the computer or holding the telephone between the neck and shoulder during a telephone conversation, can cause a headache.

To treat headaches, many people generally resort to taking various over the counter painkiller medications such as aspirin, ibuprofen or acetaminophen. However, as today's society becomes more and more health conscious and aware of natural or non-medical alternatives to treating common everyday ailments such as headaches, people are starting to consider relaxation techniques and lifestyle changes to deal with their headaches instead of ingesting a pharmaceutical product in their body. Such lifestyle changes may include getting plenty of sleep and exercise, stretching the neck and back muscles frequently, using natural, herbal, or organic remedy products, and using therapeutic aids and devices to assist in relieving tension.

Therapeutic aids or devices available in the art that are generally used to manage and provide relief from tension related pain such as headaches include therapeutic or orthopedic pillows. Such pillows are designed to provide pressure relief and/or correct body positioning when lying down by supporting and cradling the natural contours of the neck.

The problem with such therapeutic pillows is that they are not designed to provide immediate tension relief. Because body positioning is corrected over a period of time through proper placement, alignment, and support of the neck and spine during sleep, the user may not feel the therapeutic effects of the pillow until much later. Furthermore, such therapeutic pillows are not very effective in supporting the user through a wide range of sleeping positions, further delaying the desired effect of tension relief.

Traction and stretching is known to provide relief from tension by relieving pressure on individual vertebra. The dispersion of tension achieved through traction provides relief and comfort. Typically, effective traction techniques require a second person or a device to exert continuous stretching force to the target area, for example, the neck region. Disadvantageously, it may not be convenient or cost effective for a headache sufferer to immediately make an appointment to see a skilled professional to apply the required stretching force in order to seek relief. Application of traction to the neck area by a unskilled or untrained person may cause greater discomfort or aggravate the existing condition. Machinery and mechanisms used for traction are not generally readily available to the average consumer and is cumbersome and not portable for in home or office use.

Some therapeutic pillows known in the art attempt to provide traction to the cervical region. Applicant is aware of U.S. Pat. No. 4,832,007 issued on May 23, 1989 to Davis, Jr. et al. which describes a traction pillow and method. A pillow of resilient material has a generally rotatable portion (cervical roll) for supporting the cervical region of a user. With a user's neck received across the pillow's cervical roll and the user's head received on the pillow, rotation of the cervical roll in conjunction with the collapse of angled chambers defined with the resilient pillow establishes traction in the cervical region of the user.

Applicant is also aware of U.S. Pat. No. 5,542,910 issued on Aug. 6, 1996 to Oliver which describes a neck support for supporting the head and neck of a person lying on their back on a supporting surface. The neck support includes a support member having an upper surface which, when the neck support is located on the supporting surface below the head and neck of a person lying thereon, extends upwardly and rearwardly from the supporting surface to engage the back of the person's head and neck to position the head and neck at an angle.

Applicant is further aware of U.S. Pat. No. 6,006,380 issued on Dec. 28, 1999 to Sramek which describes an adjustable cervical pillow with depressions for a user's ear. The cervical pillow includes a resilient body pillow with a resilient upper portion. The top face includes a raised cervical support region for supporting the neck of a user.

The problem with existing therapeutic pillows is that none of such devices target problem areas, such as but not limited to trigger points on the head or cervical region known to be related to tension and pain. Acupressure, an ancient healing art, believes that by providing focused and direct stimulation of or pressure on the problem area known as trigger points or acupoints, user may experience greater relief more immediately and with more predictable results when the tension in such points are released. For example, and without intending to be limiting, at least three types of pain in the back of the head and neck can be attributable to the occipital nerves and associated myofascial trigger points. By stimulating and manipulating the myofascial trigger points on the neck and the underlying skin and occipital nerves, such tension and pain may be relieved.

Furthermore, none of the prior art devices provide for a portable, cost effective, and multipurpose device for relieving pain and tension that is designed for use on the head and cervical region but may be used to provide similar therapeutic effect on other parts of the body. Therefore, an unaddressed need for an improved device for relieving pain and tension exists to overcome the inadequacies and deficiencies in the prior art.

### SUMMARY OF THE INVENTION

It is an object of the present invention to provide a device for relieving pain and tension configured to target areas known to be related to the causation of such pain and tension.

It is another object of the present invention to provide a device for relieving pain and tension that simulates the feeling and firmness of fingertips applying pressure.

It is another object of the present invention to provide a device for relieving pain and tension that is portable and may be used to provide therapeutic effect on other parts of the body in addition to the neck and head area.

A device for relieving pain and tension is provided. The device includes a base having at least one pressure member support on a first side of the base wherein at least one pressure member extends upwardly relative to the pressure member support and the base, the pressure member configured to engage a user. Preferably, the base, the pressure member support, and the pressure member are made of the same resilient material and are all molded from a single piece of polyurethane gel foam.

Preferably, the pressure member support supports two pressure members positioned on the pressure member support such that the pressure members may support the head of a user while engaging the occipital region of the user's head. In an alternate embodiment of the invention, a plurality of pads positioned on the pressure member support between the two pressure members provide support to the neck such that not all the pressure is being placed on the occipital region of the user's head when the user's head is received on the device for relieving pain and tension.

In another embodiment of the invention, the base may further comprise a pair of guides positioned on opposite sides of the base to assist the user in orientating the device for relieving pain and tension for proper use.

A method of relieving pain and tension is described. To use the device for relieving pain and tension, by way of example only, in association with the head and neck region, the user would lie in a supine position on a supporting surface, use the guide on opposite sides of the base to properly position the device for relieving pain and tension on the supporting surface and under the head of the user, and position the user's head on the device for relieving pain and tension such that the pressure members engage the back of the user's head, preferably on the occipital region to engage the underlying occipital nerves.

### BRIEF DESCRIPTION OF THE DRAWINGS

The foregoing aspects and many of the attendant advantages of this invention will become better understood by reference to the following detailed description, when taken in conjunction with the accompanying drawings, wherein:

FIG. 1 is a top view of a device for relieving pain and tension;

FIG. 2 is a front view of the device for relieving pain and tension as seen in FIG. 1;

FIG. 3 is a side view of the device for relieving pain and tension as seen in FIG. 1; and

FIG. 4 is a side view of a user engaging the device for relieving pain and tension as seen in FIG. 1.

### DETAILED DESCRIPTION OF THE PREFERRED EMBODIMENT

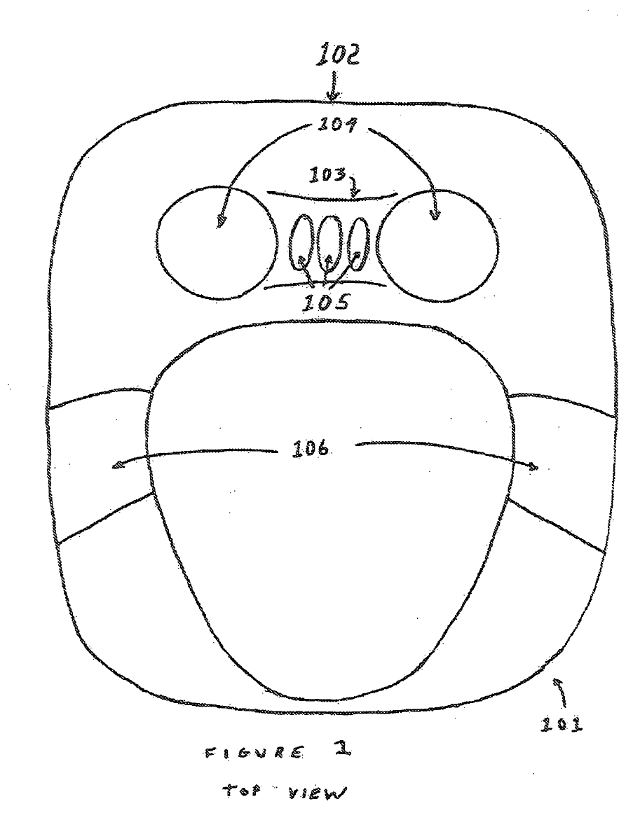

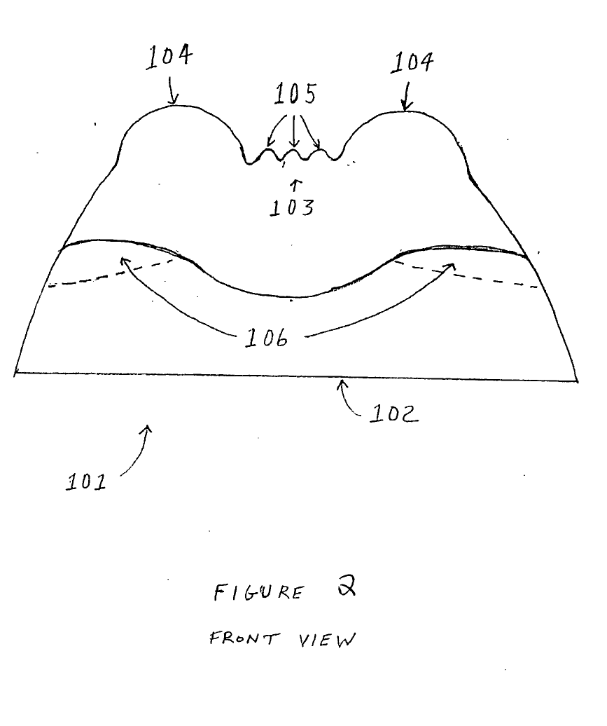

As seen in FIGS. 1 to 4, a device for relieving pain and tension headache 101 comprises a base 102 and at least one pressure member support 103 on a first side of base 102 wherein at least one pressure member 104 is positioned on pressure member support 103. When in use, pressure member 104 is configured to engage a user A.

In an embodiment of the present invention, base 102 and device for relieving pain and tension 101 is made from a resilient material such as polyurethane, plastic, or any other similar durable materials known in the art. Preferably, device for relieving pain and tension 101 is made from a single piece of semi-rigid gel foam molded to form the shape and configuration and features of device for relieving pain and tension 101 described herein and as shown in FIGS. 1 to 4. Base 102 may be made in a variety of shapes, sizes and colors so long as base 102 is capable of stabilizing and substantially inhibiting movement of device for relieving pain and tension 101 when in use. In an embodiment of the invention and without intending to be limiting, base 102 is generally rectangular in shape and approximately 6½ inches wide and 8¼ inches long.

In an alternative embodiment, device for relieving pain and tension 101 may be made in parts using a number of different materials and/or different density of materials, depending in part on cost, stability, durability and comfort required by user A and materials needed to provide such customized comfort to user A. By way of example, and without intending to be limiting, user A may desire device for relieving pain and tension 101 to have a sturdy base for maximum support but “softer” pressure members 104 due to pressure sensitivity. As such, to achieve the desired goals and specification of user A, base 102 and pressure member support 103 may be made of a firm high density polyurethane foam for greater support and durability and pressure member 104 may be made of a pressure sensitive lower density foam that will not exert too much pressure on user A when user A engages device for relieving pain and tension 101.

Base 102 comprises a first side 102a that user A engages and a second side 102b opposing first side 102a that is generally planar. Second side 102b is configured to rest on a supporting surface when in use. Preferably, the supporting surface is a generally flat and rigid surface such as the ground or a wall. However, second side 102b may engage virtually any type of surface, including but not limited to a bed, the back of a chair, or the headrest of a car seat. As described in greater detail below, when second side 102b is engaging the supporting surface, base 102 provides a stable platform for the application of pressure by user A on pressure member 104. Preferably, second side 102b is non-slip such that device for relieving pain and tension 101 may used on virtually any supporting surface

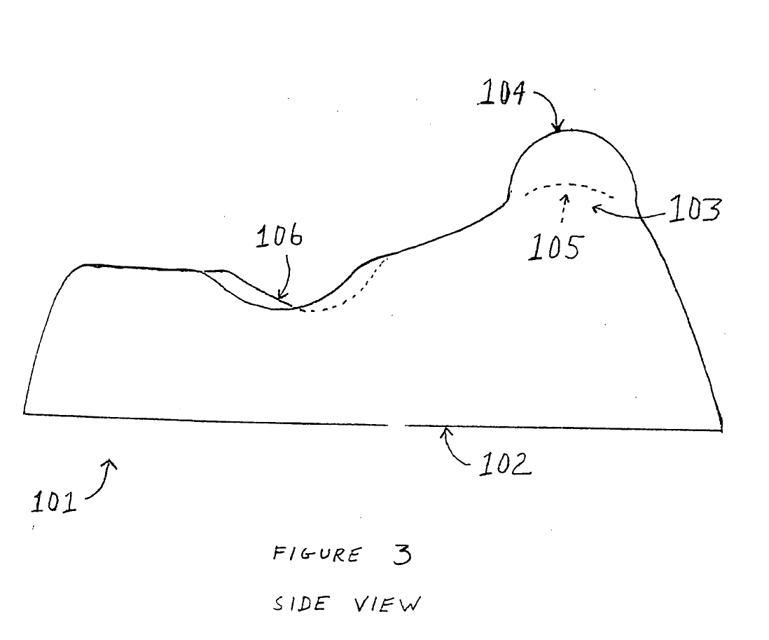

As best seen in FIGS. 1 and 3, in an embodiment of the invention, first side 102a of base 102 comprises pressure member support 103 on a first end 102c of first side 102a. Pressure member support 103 is configured to support at least one pressure member 104. Preferably, pressure member support 103 supports two pressure members 104 positioned equidistant apart, as described in greater detail below. In a preferred embodiment of the invention, pressure member support 103 is an integral portion of base 102 and made of the same resilient material as base 102, such as but not limited to polyurethane foam. Alternatively, as described above, device for relieving pain and tension 101 may be made in any number of parts using the same or different materials or density of materials, depending on the requirements of user A.

Pressure member support 103 positioned on a first end of first side 102a of base 102 is configured to support pressure member 104 such that pressure members 104 are raised above base 102 and the supporting surface which base 102 rests on when device for relieving pain and tension 101 is in use. In an embodiment of the invention, and without intending to be limiting, the height of pressure members 104 measured from base 102 to the apex of pressure members 104 is approximately 3⅜ inches. By raising pressure members 104 above base 102, pressure member support 103 creates space between pressure members 104 and the supporting surface which base 102 rests on such that user A may comfortably engage pressure pads 104. By way of example and without intending to be limiting, when device for relieving pain and tension 101 is used in association with the head and neck area, the space created by pressure member support 103 between pressure members 104 and the supporting surface which base 102 rests on enables the shoulders of user A shoulders to rest comfortably on the supporting surface while the head of user A is received on device for relieving pain and tension 101. The height of pressure members 104 measured from base 102 to the apex of pressure members 104 may be customized to accommodate the body size and shape of each individual. As described in greater detail below, when the head of user A is received on device for relieving pain and tension 101, pressure members 104 engage and apply pressure to the occipital region to provide therapeutic tension and pain relief.

Following the example of using device for relieving pain and tension 101 in association with the head and neck area, when the head of user A is received on device for relieving pain and tension 101, a second end 102d of device for relieving pain and tension 101 opposite first end 102c where pressure member support 103 and pressure members 104 are positioned may tip upwards due to the weight of the head of user A resting on device for relieving pain and tension 101. In an embodiment of the invention, additional weight may be incorporated in base 102 on second end 102d. The additional weight may be used to counter and substantially inhibit device for relieving pain and tension 101 from tipping upwards when user A engages pressure members 104.

In a preferred embodiment, pressure member support 103 supports two pressure members 104. Pressure members 104 may be made out of any resilient material, such as but not limited to polyurethane, having a range of rigidity and density to provide user A with the desired degree or level of pressure when user A engages pressure members 104. Preferably, pressure members 104 are an integral portion of pressure member support 103 and made of the same material as base 102 and pressure member support 103.

In an embodiment of the invention, pressure members 104 are positioned on pressure member support 103 such that pressure members 104 may support the head of user A while engaging the occipital region of the user's head. Pressure members 104 may be positioned equidistant apart relative to the centre of pressure member support 103 and by way of example, without intending to be limiting, pressure members 104 are approximately 2½ inches apart relative to the centre of each pressure members 104. Alternatively, pressure members 104 may be positioned anywhere along pressure member support 103 and at various distances apart from each other. In another embodiment of the invention, pressure members 104 may be adjustable in all respects along pressure member support 103, including but not limited to adjusting the distance between each pressure member 104, adjusting the angle of each pressure member 104 relative to pressure member support 103, and adjusting the rigidity of each pressure member 104, for example, by detachably attaching pressure members 104 of different rigidity onto pressure member support 103 by way of conventional fasteners such as snap fasteners. In the embodiment of the invention wherein pressure members 104 are adjustable, pressure members 104 are separate and independent parts from pressure member support 103 that may be made into various shapes and/or sizes.

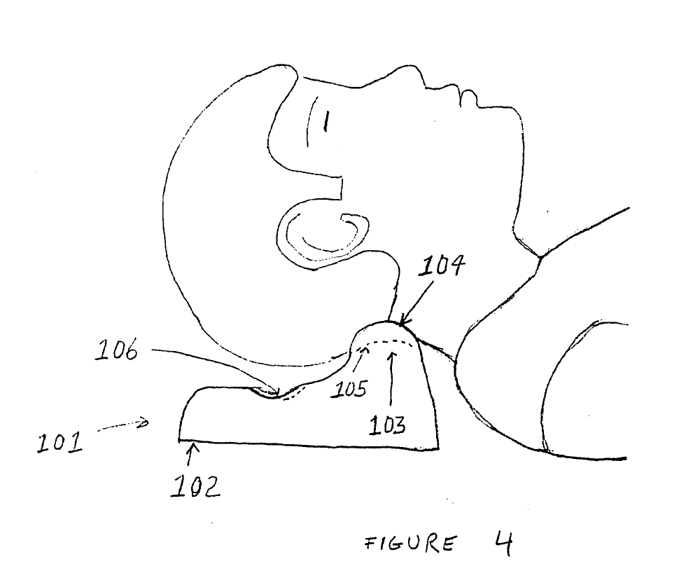

In a preferred embodiment of the invention and as seen in FIG. 4, pressure members 104 extends upwardly and rearwardly from pressure member support 103 on first end 102c and curves over at the top to extend downwardly towards second end 102d forming a “hump”. Advantageously and unlike the prior art, each pressure member 104 is a distinct member configured to engage specific trigger points on user A. As seen in FIGS. 1 and 4, when device for relieving pain and tension 101 is used in association with the head and neck area, each distinct pressure member 104 is positioned on pressure member support 103 equidistant apart relative to each other such that one pressure member 104 engages each side of the spine of user A. Pressure member support 103 is shaped such that neither the head nor the neck of user A engages pressure member support 103 when pressure members 104 engage the occipital region of the user A.

In an alternate embodiment of the invention, a plurality of pads 105 are positioned on pressure member support 103 between pressure members 104. Pads 105 are configured to provide support to the neck region such that not all the pressure is being placed on the occipital region of user A when the head of user A is received on device for relieving pain and tension 101, thus making device for relieving pain and tension 101 more comfortable for user A. Preferably, pads 105 are an integral portion of pressure member support 103 and made of the same resilient material as base 102, pressure member support 103, and pressure members 104. In an alternative embodiment of the invention, pads 105 may be made from a different material or density and may be any shape or size. In an embodiment of the invention, the position of pads 105 may be adjustable along the space between pressure members 104. In the further alternative, pads 105 may be detachably mountable onto pressure member support 103 such that user A may have the option of attaching and using pads 105 when user would like neck support or user may detach pads 105 to experience maximum pressure on the occipital region.

In another embodiment of the invention, base 102 may further comprise a pair of guides 106 positioned on opposite sides of base 102 to assist user A in orientating device for relieving pain and tension 101 for proper use. Base 102 may define a pair of indentations, one on each opposite side of base 102. Preferably, guides 106 are angled in a direction that will receive the thumbs or fingers of user A. When user A places a thumb or finger(s) into guide 106, the orientation of user A's finger or thumb in guide 106 will inform user A whether device for relieving pain and tension 101 is correctly orientated for proper use.

### Operation

By way of example and without intending to be limiting, to use device for relieving pain and tension 101 in association with the head and neck region, user A would lie in a supine position on a supporting surface, such as a comfortable mattress pad or carpeted floor. Preferably, the knees of user A are bent and the feet are generally flat on the supporting surface. User A may then grasp device for relieving pain and tension 101 with thumbs positioned in each guide 106 on opposite sides of base 102 to determine the proper orientation of device for relieving pain and tension 101 so as to correctly place device for relieving pain and tension 101 under the head of user A.

When placing device for relieving pain and tension 101 under the head of user A, second side 102b of base 102 that is generally planar should engage the supporting surface and the first end of first side 102a where pressure member support 103 and pressure member 104 are positioned should be under the neck region of user. User A should position device for relieving pain and tension 101 such that one of the two pressure members 104 are positioned on each side of the spine of user A. User A may then lower his/her head until pressure members 104 engage against the back of user A's head, preferably on the occipital region to engage the underlying occipital nerves. The occipital regions are located at the base of the skull, on either side of the spine.

When user A relaxes while his/her head is received on device for relieving pain and tension 101, pressure members 104 engage the occipital region to apply pressure to lengthen and stretch the underlying muscles causing the tension and pain, thereby stimulating blood flow to help relieve tension and tightness. User A may remain in the above described position and relax on device for relieving pain and tension 101 for a period of time sufficient to relieve the pain and tension in the neck and head area. Preferably, user A should remain on device for relieving pain and tension 101 for no more than five minutes. As user A relaxes and allows gravity to cause the head of user A to engage pressure members 104, pressure members 104 exerts an equal amount of force back on the occipital region, thereby causing the muscles to gradually relax. Eventually, the head of user A may recline back and be virtually wholly received on the second end of device for relieving pain and tension 101. However, user A should not allow his/her head to rest on the second end of base 102 for an extended period of time.

User A may similarly and conveniently, but not as effectively, use device for relieving pain and tension 101 while standing against a wall or against the head rest of a vehicle seat. User A would identify the proper orientation of device for relieving pain and tension 101 by way of guides 106 and position device for relieving pain and tension 101 in the proper orientation on the wall or the head rest such that second side 102b of base 102 engages the wall or the head rest. User A may then position his/her head and neck on device for relieving pain and tension 101. In this method of usage, user A has greater control over the amount of pressure that pressure members 104 exert on the occipital region because as user A relaxes, gravity is not causing the head to engage pressure members 104 as in the above example.

To use device for relieving pain and tension 101 in association with another part of the body such as but not limited to the lower back or the shoulder, user A may position device for relieving pain and tension 101 on virtually any supporting surface in a manner such that pressure members 104 may engage the target area. Once device for relieving pain and tension 101 is positioned in the right spot, user A my assert pressure or lean into pressure members 104 to engage pressure members 104 on the target area and wait for the muscles to release. Slight movement to massage pressure members 104 against the target area may be used.

From the foregoing description, it may be seen that the device formed in accordance with the present invention incorporates many novel features and offers significant advantages over those currently available. While the presently preferred embodiment of the invention has been illustrated and described, it is to be understood that within the scope of the appended claims, various changes can be made therein without the departing from the scope of the invention. For example, different headache reliever devices may have different acupressure pad supports, bases, comfort pads, acupressure pads of different sizes, thicknesses and dimensions and may be of many shapes and materials. These and many other features may change in different embodiments.

What is claimed is:

1. A device for relieving pain and tension, the device comprising:
(a) a base having at least one pressure member support on a first side and on a first end of said base; and
(b) at least one pressure member extending upwardly and rearwardly from said pressure member support and relative to said pressure member support;
wherein said at least one pressure member is configured to engage a user.

2. The device for relieving pain and tension of claim 1 wherein at least two pressure members extend upwardly and rearwardly relative to said pressure member support.

3. The device for relieving pain and tension of claim 2 wherein said at least two pressure members are raised relative to said base.

4. The device for relieving pain and tension of claim 3 wherein said at least two pressure members are supported on said pressure member support equidistant apart relative to each other such that one of each of said at least two pressure members may engage each side of said user's spine.

5. The device for relieving pain and tension of claim 4 wherein said at least two pressure members are detachably attachable to said pressure member support.

6. The device for relieving pain and tension of claim 4 wherein said at least two pressure members are adjustably moveable relative to said pressure member support.

7. The device for relieving pain and tension of claim 4 further comprising a pair of guides positioned on said first side of said base wherein said pair of guides are positioned on opposite sides on said first side of said base.

8. The device for relieving pain and tension of claim 7 further comprising a plurality of pads positioned on said pressure member support.

9. The device for relieving pain and tension of claim 8 wherein said base further comprises a weight at a second end opposite said first end.

10. The device for relieving pain and tension of claim 9 wherein said base, said pressure member support and said pressure members is formed from a single piece of material.

11. A method of relieving pain and tension using the device for relieving pain and tension of claim 1, the method comprising the steps of:
(a) laying in a supine position on a supporting surface;
(b) positioning said device for relieving pain and tension under a target area of a user's body;
(c) engaging said target area with at least one pressure member extending upwardly and rearwardly from said pressure member support and relative to said pressure member support

12. The method of claim 11 further comprising the step of using placing said user's thumb in a pair of guides to orientate said device for relieving pain and tension.

13. The method of claim 12 wherein said target area is a neck region of said user.

14. A method of relieving pain and tension using the device for relieving pain and tension of claim 1, the method comprising the steps of:
(a) positioning said device for relieving pain and tension on a supporting surface;
(b) positioning said device for relieving pain and tension on a target area of a user's body;
(c) engaging said target area with at least one pressure member extending upwardly and rearwardly from said pressure member support and relative to said pressure member support.

15. The method of claim 14 further comprising the step of using placing said user's thumb in a pair of guides to orientate said device for relieving pain and tension.
